(19) United States
(12) Patent Application Publication
(10) Pub. No.: US 2007/0237676 A1
(43) Pub. Date: Oct. 11, 2007

(54) Polymer micro-cantilever with probe tip and method for making same

(76) Inventors: Jonathan S. Colton, Atlanta, GA (US); Andrew W. McFarland, Orinda, GA (US); Mark A. Poggi, Atlanta, GA (US); Lawrence A. Bottomley, Lawrenceville, GA (US)

Correspondence Address: BRYAN W. BOCKHOP, ESQ., 2375 MOSSY BRANCH DR., SNELLVILLE, GA, 30078, US

(21) Appl. No.: 11/251,378

(22) Filed: Oct. 14, 2005

Related U.S. Application Data
(63) Continuation-in-part of application No. 10/414,744, filed on Apr. 15, 2003.
(60) Provisional application No. 60/372,468, filed on Apr. 15, 2002.

(51) Int. Cl. G01N 33/48

(52) U.S. Cl. 422/68.1

(57) ABSTRACT

A micro-cantilever includes a base, a micro-cantilever beam and a tip. The micro-cantilever beam extends outwardly from the base and is constructed from a material including a thermoplastic polymer material. The micro-cantilever beam has a distal end opposite the base. The tip extends transversely from the distal end. The tip is constructed from a material including a thermoplastic polymer material and is integrated with the micro-cantilever beam. To make a micro-cantilever, a mold defining a cavity having a geometry of a micro-cantilever is formed and an indentation corresponding to an image of a probe tip is formed in the mold. Molten thermoplastic is injected into the mold.

### CROSS-REFERENCE TO A RELATED REGULAR UTILITY PATENT APPLICATION

The present application is a Continuation-in-Part of, and claims priority on, U.S. patent application Ser. No. 10/414,744, filed Apr. 15, 2003, and which claimed priority on U.S. Provisional Patent Application Ser. No. 60/372,468, filed Apr. 15, 2002, the entirety of both of which are incorporated herein by reference.

### CROSS-REFERENCE TO A RELATED PROVISIONAL PATENT APPLICATION

The present application claims priority on U.S. Provisional Patent Application Ser. No. 60/620,574, filed Oct. 20, 2004, the entirety of which is incorporated herein by reference.

### STATEMENT OF GOVERNMENT INTEREST

This invention was made with support from the U.S. government under grant number IR21 EB00767-01, awarded by the National Institutes of Health. The government may have certain rights in the invention.

### BACKGROUND OF THE INVENTION

1. Field of the Invention

The invention relates to nanotechnology-based sensing systems and, more specifically, to a cantilever beam for use in analysis and atomic force microscopy and the like.

2. Description of the Prior Art

Micro-cantilevers, such as in equipment such as atomic force microscopes (AFM), are giving rise to emerging sensor platforms. The sensing mechanism is straightforward. Molecular adsorption on a resonating cantilever shifts its resonance frequency and changes its surface forces (surface stress). Adsorption onto micro-cantilevers comprised of two chemically different surfaces results in a differential stress between the top and bottom surfaces of the cantilever and induces micro-cantilever bending. Given the current imperatives to develop more sensitive and selective sensors for air-borne and water-borne toxic and pathogenic substances, rapid growth in micro-cantilever-based sensor technology is anticipated.

Micro-cantilever-based detection of airborne components is a growing application of this sensor platform. Mass sensitivities in the picogram to femtogram range are commonplace with optical reflection as the measurement mode. Chemisorption of an analyte into the coating produces a mass increase in the layer as well as a change in its interfacial stress. Thus, high sensitivity detection of individual components can be achieved by monitoring either a deflection or a shift in the resonance frequency of the cantilever. Mixture components can be qualitatively and quantitatively identified using principal component regression analysis.

Currently, the micro-cantilevers used in sensing applications are the same as those used for AFM applications. Commercial AFM cantilevers are typically fabricated from single crystal silicon, silicon dioxide or silicon nitride using conventional silicon micromachining techniques. One side of the cantilever is coated with a thin, reflective gold film to facilitate detection of cantilever deflection by optical deflection techniques. The dimensions and properties are those needed for imaging applications are far from optimal for sensing applications. In addition, the thin metal coating over one side of the cantilever renders the device extremely sensitive to small changes in temperature.

A current goal in medical diagnostic research is establishing the molecular basis of human disease. With this knowledge, the quality and duration of life will be significantly improved through prevention and early diagnosis of disease. A key factor is the development of new drugs and therapeutic monitoring systems. Mapping of the human genome was the first milestone in meeting this challenge. The second milestone lies in determining the structure and function for all proteins for which the genome encodes. While mapping of the genome is near completion, understanding of the structure and function of the proteins for which the genome encodes is in its infancy. To make full advantage of genomic information in eradicating genetically related disease states, technologies for multiplex quantification of proteins are needed, especially for monitoring of the initiation, progression and treatment of disease. Such technologies, for example, could enable profiling of tumor proteomes and improve diagnostics through analysis of constellations of proteins rather than single proteins.

Methods for multiplexed detection of proteins are more challenging than nucleic acid microarray methods due to the inherent instability of proteins, the greater variability in biophysical properties among proteins, and the lack of facile amplification and labeling methods as for nucleic acids. One microarray protein detection scheme attaches probes to a solid surface and modifies the protein analytes with fluorescent tags in order to detect them. Drawbacks of this methodology are background fluorescence and chemical modification with fluorescent tags that can block molecular recognition. The development of label-free technologies for detecting molecular interactions would be particularly advantageous.

Biosensor devices based on nanomechanical motion of micro-cantilevers comprise an emerging sensor platform having far-reaching potential in determining the molecular basis for disease. Molecular adsorption on a resonating micro-cantilever shifts its resonance frequency; the resonance shift is correlated with change in mass of the cantilever. Another more sensitive device detects the change in surface stress upon the interaction of analytes with molecules tethered to one surface of micro-cantilevers possessing two chemically different surfaces. Differential surface stress induces micro-cantilever bending which can be measured with Angstrom resolution. High selectivity in response is achievable through incorporation of biomolecular recognition elements into thin film coatings on the cantilever. Numerous micro-cantilevers having probes for a variety of proteins can be located in a single microfluidics cartridge, enabling multiplexed detection without protein tagging.

Micro-cantilever biosensors are expected to have several technical advantages over alternative sensor technologies, including greater sensitivity, less interference with the biochemical reactions and time-phased measurements of binding reactions. Since the surface area of the micro-cantilever sensor is very small (<0.004 mm²) and each molecular binding reaction contributes to the bending force on the micro-cantilever, very little analyte is required to yield a positive test indication. Since the binding reaction itself induces the bending force, intermediate steps or processes do not alter the signal. Furthermore, native molecules (i.e., not altered with a dye or tag) are attached to the micro-cantilever sensor, so there is no distortion of or interference with the desired biochemical reactions. Other advantages include: (1) The screening of multiple receptor molecules in a single fluid cell, the requirement for assaying each and every chemical for possible interaction with each and every receptor or enzyme target of interest is eliminated. With multiple target receptors arrayed in a single fluid cell or incubation chamber, faster and cheaper screening of the ever-growing number of chemicals against the many thousands of cell receptors becomes more likely; (2) Potential high sensitivity; (3) Potential high specificity; and (4) Small sample size requirement translates into less waste of expensive reagents.

Cantilevers for surface probe microscopy, such as atomic force microscopy, currently are typically made from silicon. Most have very sharp tips on the end to allow high-resolution imaging. The traditional method of cantilever fabrication begins with a silicon on insulator (SOI) wafer. This is a time consuming and expensive fabrication process that uses toxic etchants. Preparation of thin cantilevers can be quite challenging. Residual stresses produce bent cantilevers. As a result, there are a limited number of cantilever suppliers and there is little choice in cantilever geometry, flexibility and material and the resulting cantilevers are relatively costly. The most economical cantilevers are sold in wafer form (between 400-600 cantilevers per wafer). As silicon is a brittle material, one often breaks a handful of tips while trying to set up an experiment, hence raising the actual cost. Commercial silicon cantilevers are quite stiff, typically having a spring constants in the range from 0.01 to 10 N/m. This stiffness is inadequate for certain sensing applications.

For optimal performance, the stiffness of surface probe microscopy (SPM) cantilevers should be matched to the forces being measured. If a beam is too stiff, it will not deflect measurably for small forces. If the beam is too flexible, it will deflect too much or non-linearly for a large force. The stiffness of any cantilever beam results from the inherent properties of the material (for example, the Young's modulus) and the geometry of the beam. The geometry is somewhat set by the commercially available SPM and AFM equipment. For the majority of existing SPM systems, cantilever width is approximately 50 μm to maximize laser light reflection and minimize optical interference. Cantilever lengths typically range from 75 to 300 μm. The thickness is limited by processing conditions, and also by the interplay of the inherent stiffness of the material and the desired beam stiffness. Short cantilevers have recently become available. These were designed to facilitate high speed imaging and single molecule mechanical testing. Use of these requires a specialized optical detection system for measuring cantilever deflection and resonance.

The typical processing steps for fabrication of commercial cantilevers from silicon wafers include: (1) Grow 1 micron SiO₂ (both sides); (2) Grow 1 micron Si₃N₄ (both sides); (3) Pattern tip (e.g., plasma etch nitride); (4) Define tip using a dry etch; (5) Pattern and etch the cantilever using a dry, anisotropic etch; (6) Protect the tip side with polyimide; (7) Pattern and etch backside (dry etch, wet etch); (8) Remove large Si underlayer with a wet etch; and (9) Etch the middle oxide stop layer using a buffered oxide etch.

For rectangular cantilevers, the relationship between its length, l, width, w, and thickness, t, to its stiffness (spring constant, k) is given by:

$$k = \frac{{Ewt}^{3}}{4 {l}^{3}}$$

where E is the Young's modulus of the material from which it is fabricated. The relationship between the cantilever's dimension and its resonance frequency, v₀, is given by:

$${v}_{0} = \sqrt{\frac{{Et}^{2}}{4 {π}^{2} ρ}}$$

where ρ is the density of the material from which the cantilever is fabricated. Thus, the usual approach to tuning the spring constant and resonance frequency of the cantilever lies in varying its length and thickness. For sensor or soft sample imaging applications, this approach is not optimal. An additional parameter, the minimum detectable force, Fₘᵢₙ must also be taken into consideration. This parameter depends on the cantilever dimensions (through k and v₀) as well as its quality factor and viscous damping factor:

$${F}_{min} = \sqrt{\frac{2 {K}_{B} TBk}{{Qv}_{0} π}}$$

where K_{B} is the Boltzmann constant, T is the temperature in Kelvin, Q is the quality factor, B is the viscous damping factor. The traditional approach for increasing flexibility was to fabricate longer, thinner cantilevers.

Molecular interactions at or near the cantilever surface may produce an increase in cantilever mass or surface stress. Measurement of the rate and extent of these interactions are thus attainable by recording the cantilever's resonance frequency or deflection over time. If one assumes that the stress is uniformly distributed over the entire cantilever and that the response to this stress causes curvilinear deformation along its length, then the differential surface stress, Δs, is given by:
Δs=Et²[6R(1−v)]^{-1/2}

where R is the radius of curvature and v is the Poisson's ratio for the material used in cantilever manufacture. In liquid media, viscous damping decreases the amplitude of resonance, diminishing the sensitivity for detection of molecular interactions. With optical deflection techniques, measurement of cantilever deflection can be made with Angstrom level precision and, when flexible cantilevers are used, with greater sensitivity. Similarly, measurement of frequency shifts in cantilever resonance can be made with parts per billion level precision.

Cantilevers also deflect in response to changes in temperature, magnetic field strength (if coated with a magnetic material), electrostatic charge, and fluid flow. Thus, attainment of optimal sensitivity to molecular interactions at its surface requires careful design of the cantilever, reader, and sample delivery system as well as environmental control. Secondly, deflection occurs only when a sufficient number of molecular interactions result in a change in surface stress sufficient to overcome the resistance to bending (spring constant). For biosensor applications the number of molecular interactions is determined by the surface area of the active side of the cantilever, the number of covalently attached probe molecules, and the entropic impact of this interaction. Thirdly, the temporal response of cantilever deflection is limited by the rate of mass transport of the target to the probe. Transport of material to the cantilever surface can be achieved by convection, migration (or polarization), and diffusion. Minimization of sample volumes needed for analysis and cantilever response to changes in fluid flow rate (i.e., convection) and migration (i.e., electrostatics), necessitates small cell volumes and diffusion-based transport. Thus, optimization of the analytical sensitivity for micro-cantilever-based immunoassays requires careful consideration of the following interdependent factors: (1) Cantilever spring constant; (2) Surface area of the active element; (3) Sample and cell volumes; (4) Spatial distribution and orientation of the probe on the surface; (5) Affinity of all surfaces for non-specific binding of target and matrix components; (6) Optical gain; (7) Positional sensitivity of the reader.

Photopolymer-based SPM cantilevers have been previously proposed. These were developed for internal, laboratory use and have not been produced in large, economic and commercial quantities. All were produced using microelectronics manufacturing techniques, requiring expensive tooling housed in a clean room environment. In one example, cantilevers were produced from an epoxy-based photopolymer, a photoresist material of the type used in microelectronics processing that is quite brittle and that may not be suitable for many sensing applications. Photopolymers react (cure) when exposed to light, so one creates the required cantilever shape by exposing the photopolymer to patterned light. The tips were placed on the cantilever using electron beam deposition (EBD), which is not well suited for mass production. Reactive ion etching and photopolymer have been used to create cantilevers and tips. In another example polymer cantilevers were produced from SU-8, a epoxy-based photopolymer used as a photoresist in microelectronics manufacturing. A silicon mold, produced using traditional isotropic and anisotropic etching techniques, could be cleaned and reused. The cantilever, tip and chip was fabricated by sequentially spin coating SU-8 onto the wafer, photolithographically crosslinking the polymer followed by rinsing and thermal curing of the crosslinked SU-8. A major challenge was control of the cantilever thickness, a critical parameter in determining cantilever stiffness.

In another example cantilevers were formed from fluoropolymers to produce cantilevers and structures for bio-micro electronic mechanical systems (MEMS). The goal was to produce cantilevers that can be biochemically functionalized for AFM. These were manufactured by ion beam etching, a complicated and expensive process that is not amenable to mass production. To date, polymer cantilevers have been produced using techniques that facilitate formation of sharp tips on their ends for imaging applications. For most sensing applications, a tip is superfluous. Also, the methods of fabrication inherently limit the number of polymeric cantilevers that can be produced at one time. It is clear that more robust, more economic cantilevers with variable mechanical properties and improved biocompatibility would be a boon to the field of scanning probe microscopy and micro-cantilever sensors.

Injection molding is by far the most used to mass-produce complex, three dimensional thermoplastic polymer products. Micromolding, the molding of polymer parts with dimensions on the order of 100s of μm and features on the order of 5-10 μm, is just now coming into commercial use. Injection molding machines capable of producing such parts are now commercially available. This technique has the potential for extremely high throughput. In traditional injection molding, cycle times are on the order of tens of seconds, leading to a time per part of seconds or less for multiple cavity molds. The most complicated have 144 cavities, but most have tens of cavities. As a result of these mass production techniques, polymer parts are quite cheap, with a rule of thumb being two to three times the cost of the material used to make the part. In addition, injection molding is a very well understood and controllable process. Therefore it repeatably produces high quality parts, at a very high level of performance.

In the fabrication of microfluidic devices, such as flow chambers for micro-electrophoresis, hot embossing is a commonly used technique. This technique presses a hot mold (which is a negative of the part desired) into a polymer sheet. Common polymer materials used include PMMA and polycarbonate (PC). Polystyrene is a thermoplastic polymer with a long history of use in medicine, biochemistry and molecular biology. Plastic parts (e.g., microtiter plates and Petri dishes) are made from polystyrene by heating it to soften it, forcing into shape by injecting it into a mold, cooling it to harden, and then removing it from a mold. There are no chemical reactions, hence the process is quite clean and environmentally friendly. Mass production of polymer products from polystyrene is widely practiced and very well characterized polymer processing field.

The scanning tunnelling microscope (STM), invented in 1982, revolutionized the microscopy world by enabling sub-Å resolution, allowing true atomic scale exploration of samples. In 1986 the atomic force microscope (AFM) and the scanning force microscope (SFM) processes were introduced. Essentially these approaches used an STM to detect the deflection of a micro-cantilever with an asperity-like tip, which was scanned over samples to reconstruct surface topography. One advantage of the atomic force microscope is that nonconductive samples could be interrogated, a process that is impossible with the STM.

Since 1986, the term ‘atomic force microscope’ has grown to describe an apparatus that measures the deflection or resonance behavior of a micro-cantilever by numerous means (e.g., optical lever, capacitance, piezoresistance, and piezoelectric). Regardless of the motion detection scheme, there are two modes of micro-cantilever operation: static (or DC) and dynamic (or AC). The static mode allows for determination of surface stress or surface topography, for example, while the dynamic mode allows for determination of adsorbed mass. By employing these two operational modes and one of the motion detection schemes, atomic force microscopes have been used to investigate an impressive and expansive array of scientific fields from calorimetry and rheology to biological adsorption events.

Most current SFM approaches employ tipped micro-cantilevers which are fabricated using integrated-circuit (IC) manufacturing techniques (e.g., lithography, etching, and deposition) and, almost exclusively, they are made from silicon or similar materials (e.g., SiN). The IC-based approach limits the material properties (e.g., elastic modulus and density) of feasible micro-cantilevers as well as their methods of production. Additionally, the IC approach is expensive (mainly due to the need for clean rooms), pushing the cost of a single SFM micro-cantilever part (usually comprising one to five micro-cantilevers) from five to over 100 dollars.

To expand the array of feasible materials for micro-cantilevers, one method employed IC techniques to make tipped micro-cantilevers from the photopolymer SU-8 for scanning force and scanning near-field optical microscopy. Another method used similar techniques to produce polyimide micro-cantilevers with PDMS tips for microscopy and contact printing. Others have produced tipless micro-cantilevers from polymeric materials using IC-based approaches, solvent casting, and injection molding, microstere-olithography, and multi-photon polymerization-based approaches—tipless micro-cantilevers are used mainly in chemical and biological sensing applications.

In terms of these nonceramic micro-cantilever production approaches (both tipped and tipless), the IC-based techniques are extremely scalable and produce cantilevers that are closer to an ideal parallelepiped than the other methods, and submicron thickness cantilevers are possible (thinner cantilevers exhibit superior deflection sensitivity). However, the initial set-up costs are very high (due the need for a clean room) and the number of feasible materials is very limited. The solvent casting approach cannot yet yield tipped probes, but has the ability to produce submicron thickness polymeric cantilevers and potential for scalability could be present. Injection molding has reasonable scalability potential, production capabilities using many different types of polymeric materials (e.g., amorphous, semi-crystalline, and fiber reinforced), and the set-up cost is much less expensive than IC-based approaches. However, injection molding cannot yet produce cantilevers of the same geometric caliber as the IC approaches (i.e., injection-molded parts are less close to ideal parallelepipeds). The microstereolithography and multi-photon polymerization-based approaches are very appealing for cantilever-based micro-fluidic applications because the fluid cell could possibly be built around the cantilever itself; however their scalability has yet to be demonstrated and initial set-up costs could be high. Also, the multiphoton polymerization-based approach can produce cantilevers with submicron thickness.

There is also a need for a micro-cantilever having a probe tip that can be mass-produced inexpensively.

### SUMMARY OF THE INVENTION

00371 The disadvantages of the prior art are overcome by the present invention, which, in one aspect, is a micro-cantilever that includes a base, a micro-cantilever beam and a tip. The micro-cantilever beam extends outwardly from the base and is constructed from a material including a thermoplastic polymer material. The micro-cantilever beam has a distal end opposite the base. The tip extends transversely from the distal end. The tip is constructed from a material including a thermoplastic polymer material and is integrated with the micro-cantilever beam.

In another aspect, the invention is a method of making a mold for a micro-cantilever. A geometry for a micro-cantilever beam having a distal end is selected. A mold defining a cavity having the geometry of the micro-cantilever beam is formed. An indentation, corresponding to an image of a probe tip, is formed in the mold at a selected location corresponding to the distal end of the micro-cantilever beam.

In yet another aspect, the invention is a method of making a micro-cantilever in which a molten thermoplastic is injected into a mold. The mold defines a cavity corresponding to a geometry of a cantilever beam having a distal end. The mold also defines an indentation disposed adjacent the distal end. The indentation corresponds to a geometry of a probe tip. The mold is allowed to cool so as to allow the molten thermoplastic in the cavity to substantially solidify, thereby forming a cantilever beam with a probe tip. The cantilever beam with the probe tip is removed from the cavity.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a plan view of one embodiment of a micro-cantilever and a base, according to the invention.

FIG. 2 is a plan view of a mold according to one embodiment of the invention.

FIG. 3A is a side cross-sectional view of an injection molding machine and a mold prior to injection of a material into the mold.

FIG. 3B is a side cross-sectional view of an injection molding machine and a mold after injection of a material into the mold

FIG. 4A is a schematic diagram of a reactive treatment applied to a micro-cantilever beam prior to reaction with a reactant.

FIG. 4B is a schematic diagram of a reactive treatment applied to a micro-cantilever beam after reaction with a reactant.

FIG. 5 is a schematic diagram of a micro-cantilever beam and a deflection detector.

FIG. 6 is a block diagram of an analysis system according to one embodiment of the invention.

FIG. 7 is a side view of a micro-cantilever beam with a tip.

FIG. 8 is a side view of a micro-cantilever beam that including an optical channel.

FIG. 9 is a side view of a micro-cantilever beam including reinforcement.

FIG. 10 is a plan view of a micro-cantilever beam having a specific geometry.

FIG. 11 is a side cross-sectional view of an injection molding machine and a mold with an integrated probe tip image.

FIGS. 12A-12C are detail side cross-sectional views showing formation of an integrated probe tip image in a mold.

FIG. 13 is a micrograph of one example of a probe tip on the end of a cantilever.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Unless otherwise specified herein, the drawings are not necessarily drawn to scale.

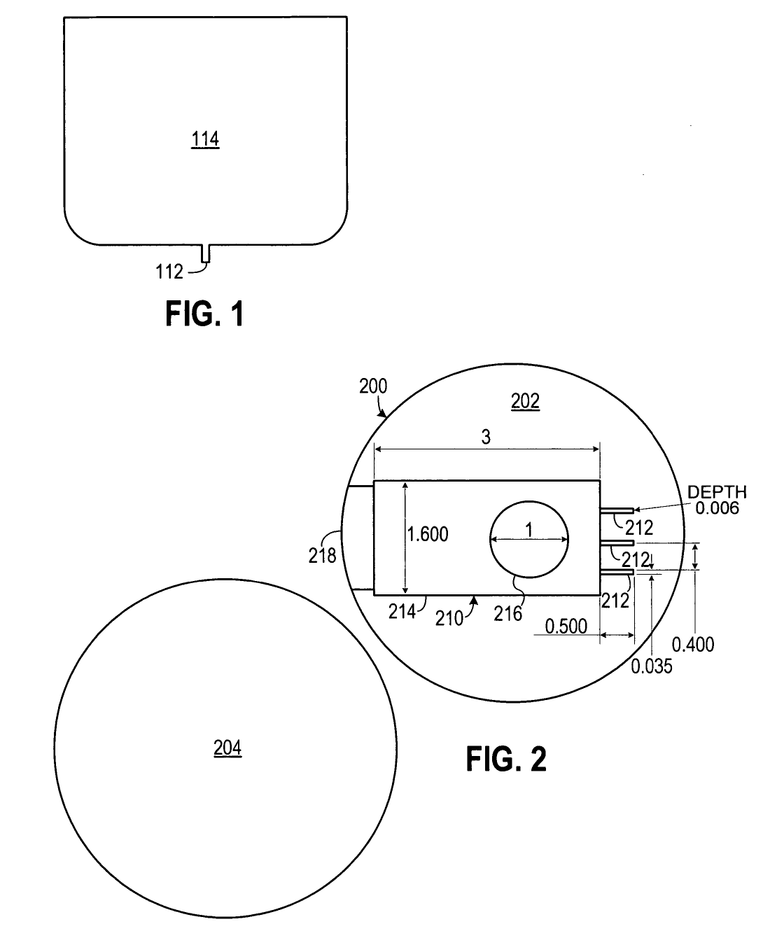

As shown in FIG. 1, a micro-cantilever beam 112, according to the invention, is made from a thermoplastic polymer material. The micro-cantilever beam 112 could extend outwardly from a base 114 that can also be made from the thermoplastic material. The micro-cantilever beam 112 could be manufactured through one of several processes employed in micro-fabrication, including: injection molding, shaping a fiber, cutting a pre-shaped fiber to a predetermined length, casting by placing an uncured polymer into a mold and allowing the polymer to cure, and cutting the micro-cantilever beam 112 from a sheet of thermoplastic of a suitable thickness. The thermoplastic resin could include: polystyrene, polypropylene, polyethylene, acrylonitrile butadiene styrene, polycarbonate, PMMA, polyester, polyimid, liquid crystal polymer, or polyamide, or a combination thereof Using a thermoplastic in manufacturing the micro-cantilever beam 112 offers the advantage of decreased cost of manufacturing (the cost of manufacturing en masse being on the order of several times the cost of the raw thermoplastic material being used) and the added utility of being able to affix reactants directly to the micro-cantilever beam 112 without applying another material, such as gold, first.

Two alternate techniques that may be used for mass production of a micro-cantilever beam include hot embossing, and laser ablation. Hot embossing is a process whereby a die in the shape of the part is heated and stamped into a polymer film. This process is similar to coining in metals, in that very little material is displaced and very fine and accurate features can be produced. A die could be manufactured in the shape of a comb, using any number of manufacturing techniques, such as micro-EDM, electrochemical machining, laser machining, or reactive ion etching. This die then would then be stamped into a very thin film of the polymer of interest, forming the cantilevers by melting away the unwanted material. Laser ablation could be used to form the cantilever tips directly from sheets of polymer. A laser could be directed to remove (melt) material and hence form the tips. One advantage of this process is the width of the laser beam only needs to be on the order of the size of the gaps between the cantilevers, not the cantilever itself An eximer laser would be suitable for this process.

As shown in FIG. 2, (all dimensions in FIG. 2 are given in millimeters) a mold 200 may be used to manufacture the micro-cantilever beam. The mold 200 shown in FIG. 2 could be used to manufacture a plurality of micro-cantilever beams simultaneously. The mold 200 could include a plate member 202, typically made from a suitable metal, such as steel. A cavity 210, defined in the plate member 202, includes one or more micro-cantilever beam-shaped cavities 212 extending outwardly from a base cavity 214. An injection port 218 allows material to be injected into the cavity 210 and a hole for a knock-out pin 216 facilitates removal of the micro-cantilever beams from the mold 200. The cavity may 214 be formed in the plate using one of several methods, including: micro-electrical discharge machining; LIGA; etching; machining; laser ablation; or electro-chemical machining. A base member 204 is placed against the plate member 202 form the bottom of the cavity 210.

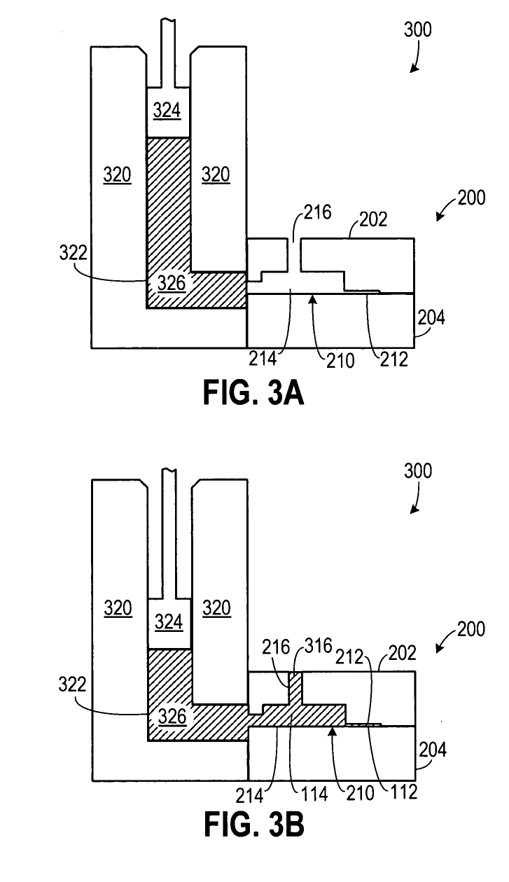

An injection molding system 300 is shown in FIGS. 3A and 3B. In such a system, the mold 200 is placed against an injector 320 that includes a thermoplastic material 326 well 322 and a piston 324 for extruding the thermoplastic 326 from the well 322 into the cavity 210 defined by the mold 200. Typically, a heater (not shown) is included with the injection molding system 300 to melt the thermoplastic material 326 and heat the mold 200.

Injection molding may be accomplished through the use of a NanoMolding machine (such as model Sesame .080, from Murray Engineering, Buffalo Grove, Ill.). Such a machine is capable of injecting milligrams of material at a time. The NanoMolding machine is a dual plunger system. A vertical plunger melts the thermoplastic pellets by forcing them through a heated capillary. This creates the shot in front of the injection plunger. The injection plunger then forces the plastic into the empty cavity, thereby producing the part. The part cools and is removed.

Injection molding allows the making of many identical cantilevers. Typical injection molds for commercial applications contain multiple cavities. This allows for a higher utilization of the injection molding machine and mold.

In one example of a cantilever beam manufactured according to the invention, cantilever beams without tips had initial dimensions as follows: 500 μm long, 50 μm wide and 5 to 10 μm thick. This resulted in cantilevers with moduli in the 5-10 mN/m range.

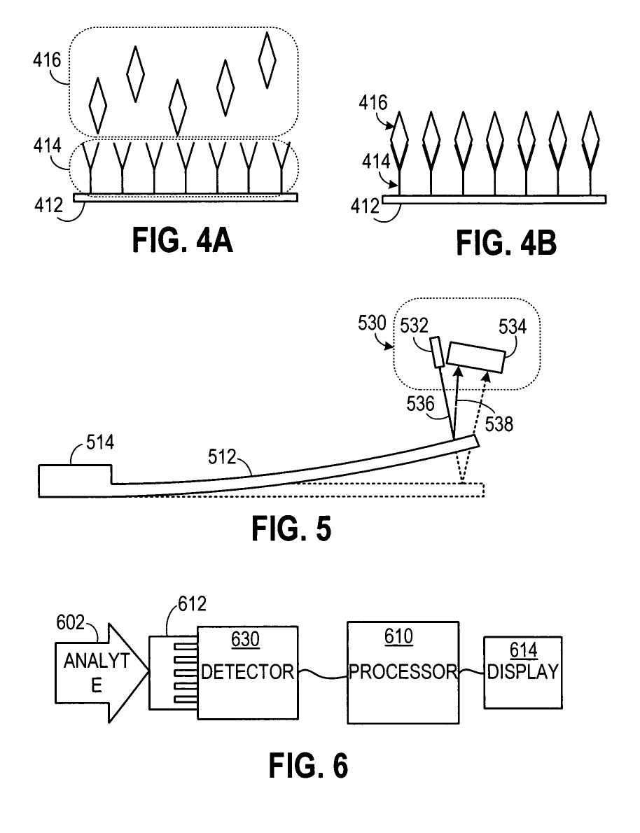

Micro-cantilever beams, according to the invention, could be employed in analysis and sensing systems, to perform such tasks as bioassay and molecular assay analysis. As shown in FIGS. 4A and 4B, in one embodiment, the micro-cantilever beam 412 could include a reactive treatment 414 that is applied to a selected side of the micro-cantilever beam 412. The reactive treatment 414 will cause the micro-cantilever beam 412 to exhibit a predetermined change in a physical property (such as deflection or frequency response) in a first manner when the reactive treatment has not reacted with a selected substance 416 (as shown in FIG. 4A). The reactive treatment 414 causes the micro-cantilever beam 412 to exhibit the physical property in a second manner, different from the first manner, when the reactive treatment 414 has reacted with the selected substance 416.

The composition of the reactive treatment 414 would depend upon the substance being sensed. For example, in an immuno-assay, the reactive treatment 414 could include an antigen that is receptive to an antibody. In an assay of an infectant, the reactive treatment 414 could include an antibody that is receptive to an antigen or some other protein associated with the infectant. Similarly, the reactive treatment 414 could include a treatment used in molecular recognition, a treatment used in biological recognition, a treatment used in bio-molecular recognition, or one of many other types of reactive treatments used to recognize substances.

While not shown, the micro-cantilever beam 412 could include two different reactive treatments (or the same reactive treatment is different concentrations) applied to opposite sides of the micro-cantilever beam 412 to derive information, for example, about the proportion of one substance to another substance.

The physical properties associated with the micro-cantilever beam 412 could include deflection of the micro-cantilever beam 412 from a non-reactive state and a change in frequency response.

Sensing the state of the micro-cantilever beam 512 is shown in FIG. 5. One end including a beam holder 514 of the micro-cantilever beam 512 is secured to a stable platform (not shown), while the other end is allowed to move freely in at least one axis. A beam state detector 530 includes a light source 523 that generates a laser beam 536 that reflects off of a predetermined spot of the micro-cantilever beam 512. The reflected beam 538 then is sensed by a photo-detector 534. The amount of deflection could be determined by measuring the displacement of the reflected beam 538 or through the use of interferometry. While FIG. 5 shows one example of a micro-cantilever beam state sensor, many other types of micro-cantilever beam state sensors are available and would be suitable for use with embodiments of the invention.

A system for detecting several substances in an analyte 602 (or for quantifying a concentration of a single substance) is shown in FIG. 6. The system includes an array of micro-cantilever beams 612, around which the analyte 602 is passed. Each micro-cantilever beam of the array 612 could be treated with a different reactive treatment to detect different substances in the analyte 602 or could be treated with the same reactive treatment in differing densities to detect a concentration of a single substance in the analyte 602. A detector 630 senses the state of the micro-cantilever beams 612, once the system is in steady state, and transmits the state information to a processor 610. The processor 610 determines the presence or concentrations of the substances being assayed and the resulting information is output to a user interface, such as a display 614.

In one specific embodiment, the detector for multi-micro-cantilever could include 670 nm diode lasers, a fluid cell and quadrant photodiode position-sensing devices (PSD) (available from Pacific Sensor Inc.). Laser light is reflected off the reflective side of the cantilever onto the PSD. The voltage output of the PSD is proportional to the magnitude of cantilever deflection whereas the sign is indicative of the direction of bending (up or down with respect to a metal-coated side). The voltage output of each PSD is amplified (each PSD is mounted on its own amplifier card) and sent to a Signal Analyzer (such as a Model 785 Dynamic available from Stanford Research Systems). Frequency shifts in cantilever resonance may be measured by taking repeated Fourier transforms of the time dependent PSD. Frequency versus time data may then be downloaded to a computer for display. Cantilever deflection measurements may be recorded by taking the voltage output of each PSD (normalized for changes in diode laser output) as a function of time by downloading this data directly to a laboratory computer for display.

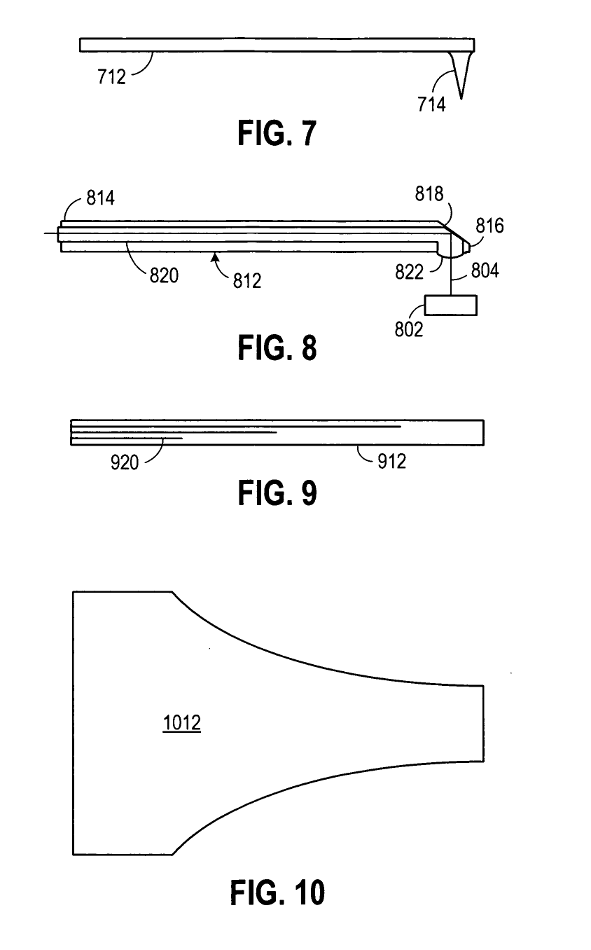

A tip 714 may be added to the micro-cantilever beam 712, as shown in FIG. 7. Addition of a tip 714 could be performed in one of several ways, including: molding the tip 714 onto the micro-cantilever beam 712 as part of a molding process, thereby making an integrated beam and tip unit; gluing the tip 714 to the beam 712; heating the tip 714 and melting it into the beam 712; or one of many other methods of attachment. Use of the tip 714 could allow the micro-cantilever beam 712 to be used in such applications as atomic force microscopy (AFM) and dip pen lithography. The tip 714 could be a silicon AFM-type tip, a plastic tip, a carbon nanotube or one of many other types of tip.

A micro-cantilever beam 812 according to the invention, as shown in FIG. 8, could include an embedded optical channel 820 extending from a proximal end 814 to a distal end 816 of the beam 812. The optical channel 820 could terminate in a lens 822 and include a reflector 818. The optical channel could be used to guide an electromagnetic beam 804 to and from a surface 802 being imaged. The optical channel 820 could also be used to deliver a light beam to a preselected spot for such applications as micro-laser ablation. A change in the geometry of the optical channel 820, indicating a deflection of the micro-cantilever beam 812, could be sensed through interferometry.

As shown in FIG. 9, an additive, such as a reinforcing agent, could be added to the thermoplastic material of the micro-cantilever beam 912 so as to modify a mechanical figure of merit of the micro-cantilever beam 912. For example strips 920 of a different substance than the thermoplastic material could be embedded in the micro-cantilever beam 912 along one or more preselected planes to modify stiffness and linearity of response. Also, other additives could be used, including: nanotubes, nanoparticles, nanofibers, microtubes, microparticles, microfibers, tubes, particles, or fibers. Additives could also be added to modify other physical properties, including: electrical conductivity; frequency response; minimum force required to deflect and many other properties that facilitate use of the invention in specific applications.

As shown in FIG. 10, alternate geometries of the micro-cantilever beam 1012 may be used to further refine the physical characteristics of the micro-cantilever beam 1012. The geometry could include several dimensional factors, such as: length, width, height and shape. For example, a geometry that gives the micro-cantilever beam 1012 a linear response to a linear force applied thereto could be selected. The specific thermoplastic material, possibly in combination with reinforcing agents, could be selected to tune the physical behavior, e.g., stiffness, frequency response, linearity of deflection, minimum force required to deflect the beam, of the micro-cantilever beam 1012.

Young's modulus of Polystyrene (approximately 3 GPa) makes it ideal for use as a SPM cantilever probe. Other polymers, such as polymethylmethacrylate (PMMA), polycarbonate (PC), polyethylene (PE) and polypropylene (PP) are also thermoplastics, having widely varying moduli (stiffness) and bio-chemical compatibility. As a result of their different moduli, varying stiffness cantilevers could be manufactured with the same geometry. The latter is important so that the cantilevers will be compatible with commercial SPM equipment (will fit them without modification). Another way to vary the modulus of a polymer material is to add reinforcements in the form of particles or fibers, hence forming a composite. Nano-clays or nano-fibers could be added in very small levels to polystyrene to change its mechanical properties, without affecting its bioadhesion. As a result, one could tune the mechanical properties of a cantilever, without changing its geometry, which is important for assuring its compatibility with existing SPM equipment.

One can design to the desired stiffness of the cantilever by varying the basic geometry (length, width, thickness), of course within limits circumscribed by compatibility with existing equipment. Once the mold has been fabricated to specified dimensions, the spring constant of the cantilever can be tuned by varying the polymer composition. This manipulation would produce tunable spring constants, and hence allow one to vary its sensitivity as a sensor. The addition of fillers, such as nano-scale particles (clays), fillers (carbon, glass, ceramic, polymeric, metallic), and fibers (carbon, glass, carbon nanotubes, metallic, ceramic), hence forming composites and functionally gradient materials, allows the stiffness of the base material to be varied by orders of magnitude, without changing the chemical properties of the surface of the cantilever. The geometry of the cantilever also could be varied to accomplish tunable spring constants. For example, the beam's width could taper towards it end, which would give interesting mechanical response, such as a constant spring force regardless of deflection.

The following table relates various polymer materials to other cantilever beam physical properties:

| | | Cantilever | |
|---|---|---|---|
| | Young's | stiffness | Cantilever |
| | modulus | (50 μm | stiffness (35 μm |
| Material | (GPa) | wide) (mN/m) | wide) (mN/m) |
| polystyrene | 3.0 | 37.5 | 26.3 |
| polycarbonate | 2.4 | 30 | 21 |
| polymethylmethacrylate | 2.6 | 32.5 | 22.8 |
| polyethylene | 1.1 | 13.8 | 9.7 |
| polyproplylene | 1.3 | 16.3 | 11.4 |
| ABS | 2.4 | 30 | 21 |
| polytetrafluoroethylene | 0.55 | 6.9 | 4.8 |
| nylon 6 with 1.6 vol % | 2.1 (1.1) | 26.3 (13.8) | 18.4 (9.7) |
| nanoclay filler (neat | | | |
| nylon 6) | | | |

As one can see, a wide variety of stiffness can be obtained by changing the material, the dimensions or both. These materials also cover a wide range of reactivity with biological materials, allowing one to select.

The injection-molding approach to tipped cantilever fabrication holds promise due to the ability to employ literally hundreds of thermoplastic polymers to tailor the physical (via material elastic modulus and micro-cantilever geometry) and chemical (via material type) properties of arbitrarily shaped cantilevers and their features at vastly reduced cost. Compared to silicon-type parts, different polymers would show different tip-sample van der Waals (VDW) interaction forces (for a given sample surface); experimental investigation of these forces would be difficult (and could be impossible) with commercial, nonpolymeric probes. Additionally, these altered VDW forces which are tenable with polymeric probes could offer experimental advantages in certain situations (e.g., higher-quality noncontact mode operation with lowered VDW interactions). Due to the vast array of injection-moldable polymers, the experimental space of tip-sample surface chemistries is greatly expanded with the injection-molding approach. For example, it would be difficult if not impossible to employ a silicon-type probe to investigate interaction forces between a polymeric probe tip and polymeric surface, say polystyrene (PS), but with a PS probe this becomes trivial.

Tipped polymeric micro-cantilevers could be used in fields outside the scanning force field as well. Tribological applications such as investigating attrition-type tip wear under tip-sample contact and relative movement are possible. Micro-cantilever tip functionalization could be vastly simplified with polymeric parts for force spectroscopy applications—antibodies (and antigens) will spontaneously bond to PS under incubation possibly enabling experimental investigation of the rupture forces between antibodies (or antigens) and (non)functionalized surfaces. It is not implied here (nor should it be inferred) that polymeric parts are superior in all applications, only that there could be applications for which polymeric parts show certain advantages.

The invention, in one exemplary embodiment, expands upon tipless plastic cantilevers for biosensing applications, to injection mold polymeric micro-cantilevers with integrated tips. These tipped cantilevers may be used to image repeatedly a silicon step-height grating to examine wear resistance; the grating also may also be imaged using a commercially available AFM cantilever probe to assess the quality of the image produced by the plastic probe. The injection molding process consists of forcing a molten thermoplastic polymer into a hollow cavity, allowing the polymer melt to cool and harden in the cavity, and finally removing the completed part from the cavity.

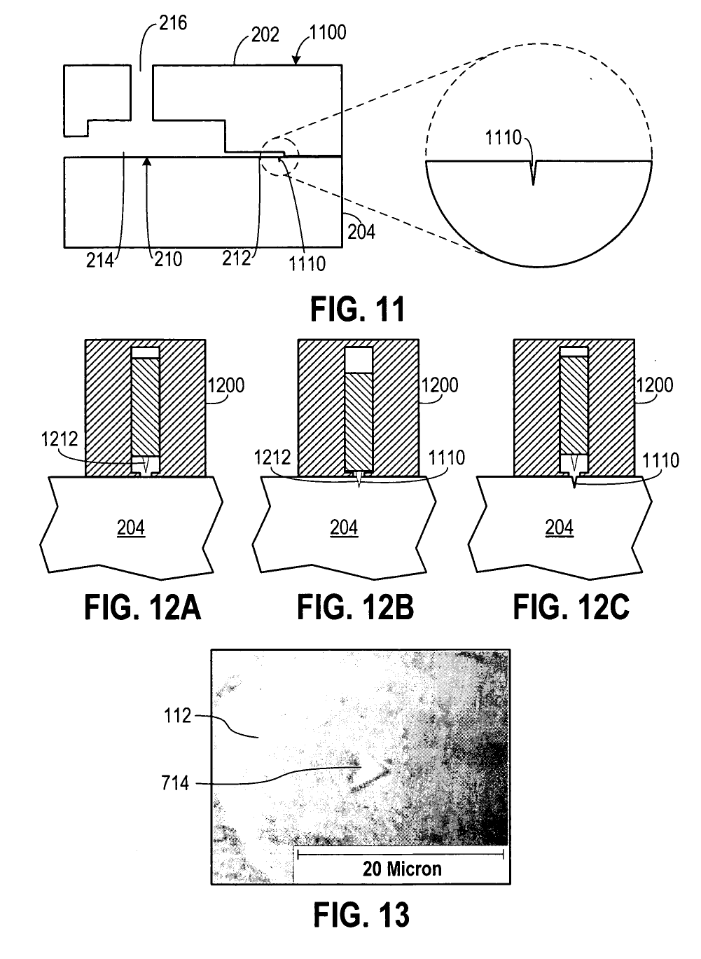

A mold 1100 for forming a tipped micro-cantilever is shown in FIG. 11. It is essentially the same as the mold shown in FIG. 3A, except that includes an indentation 1110 corresponding to the shape of a probe tip. In creating the mold 1100, as shown in FIGS. 12A-12C, a nanoindenter 1210 may be used to punch the indentation 1110 into the plate member 202 or the base member 204 at a location corresponding to the desired location of the probe tip. The nanoindenter 1210 includes a tip 1212 that could be made from a diamond. The tip 1212 is driven toward the portion of the mold where the indentation 1110 is to be placed, as shown in FIG. 12B, and then removed, as shown in FIG. 12C—leaving the indentation 1110.

In one experimental method employed to make an integrated-tip cantilever, the mold consisted of two rectangular steel blocks, one of which contained the cavity forming the desired part, while the other contained a ‘sprue,’ the avenue delivering the polymer melt from the barrel of the injection-molding machine to the cavity. The mold cavity was formed by two machining processes, the first of which used a 0.8 mm diameter end mill (operated at 5000 RPM, with a transverse feed rate of 1 mm per minute, a plunge feed rate of 0.1 mm per minute, and a plunge step size of 100 μm) to produce a large base part cavity. The portions of the cavity which form the micro-cantilevers were then cut with a 50 μm diameter end mill (operated at 50 000 RPM, with a transverse feed rate of 0.5 mm per minute; no material was removed on the plunge). To produce the portion of the cavity used to form the SFM tip, a Nanoindenter XP was used (available from MTS Systems, Eden Prairie, Minn.) with a Berkovich (three-sided pyramidal) tip. The radius of curvature of this tip was approximately 40-60 nm and the indentation depth was approximately 1.5 μm. This type of tip is not similar to the asperity-like tips present on silicon SFM micro-cantilevers; nonetheless, this tip geometry proved feasible. The overall shape of the tip might be modified if the cavity were made with other known techniques, such as focused ion beam milling. Once completed, the mold halves were mounted in a Sesame.080 Nanomolder (available from Medical Murray, Buffalo Grove, Ill.), a machine geared toward the production of small volume parts. The mold was heated to 175° C. prior to injection and the polymer melt to 205° C. The mold heat supply was shut off immediately after the cavity was filled (i.e., at the end of injection). The injection was pressure limited at 50 MPa, and the total injection time was approximately 1 second. The holding time was set at 30 seconds, and then fluid (water) cooling was active for 15 seconds after the holding period. At this time the mold halves were separated and the part removed manually. Twenty-five parts were made in this experiment. As a note, economic high-volume production would be feasible using this apparatus with a cycle time of approximately one minute.

A micrograph of a polymeric tip 714 and a cantilever 112 made in this experiment is shown in FIG. 13. The polymer used was polystyrene. The dimensions of the micro-cantilevers were roughly 250 by 75 by 10 μm (length-width-thickness). The first-mode bending resonant frequency (obtained via a curve fit to thermal spectra data) and bending stiffness (obtained with the method of Hutter and Bechhoefer) of the part is 56.4 kHz and 4.2 N per meter, respectively.

A CSC 12 Tapping Mode™ cantilever (MikroMasch, first bending mode resonant frequency of 360 kHz) was used to image the polymeric tip, which showed that the tip region of the mold is completely filled. Both the drive amplitude of the imaging cantilever and the set-point values were optimized so as to minimize distortions of the polymer tip during imaging. From this image, the radius of curvature of the polymeric tip was estimated to be roughly 62 nm, which is reasonable considering the 40-60 nm radius of curvature of the mold indentation which forms the micro-cantilever tip.

The imaging quality of an injection-molded AFM probe was compared to that of a commercially available MicroleverTMAFM probe (Part #MLCT-AUHW, Veeco Metrology), which had a spring constant of approximately 0.03 N per meter and a tip radius of curvature of approximately 15 nm. A custom-made silicon step-height grating was used as the test specimen as its sharp edges provide well defined features. The grating was interrogated in the AFM's contact mode with a polymeric micro-cantilever and in the tapping mode with the commercial probe (Microlever). Tapping mode usually produces higher quality images, hence it was used with the silicon part to obtain a higher quality picture for comparison to the contact mode, polymeric-probe-obtained image. This is a conservative approach to ensure that any experimental agreement between the commercial and polymeric probes would be due to the cantilevers themselves and not the measurement mode. The rounding induced from scanner movement was removed from all AFM images by performing a first-order plane fit using the AFM (Nanoscope IIIa, available from Veeco Instruments) software. Using a profilometer (Alpha-Step 500, available from KLA/Tencor), the step height of the grating was measured to be 1.06 μm. Prior to image acquisition the AFM was toggled into ‘Force Curve’ mode so that a minimum contact force could be established, which reduced the likelihood of excessive polymeric tip wear. The image obtained by the silicon part showed an average step height of 1.018 μm, while the image obtained by the PS part showed an average step height of 1.057 μm. This is approximately a 4% difference, and indicates that the PS tipped-cantilevers are feasible for SFM. In addition, the equivalence of image quality is even more impressive as the plastic tip was operated in the ‘contact’ mode while the silicon tip was operated in the ‘tapping’ mode, the latter of which is generally considered to produce higher quality images. The agreement between the step heights that were measured with each probe type (i.e., silicon and plastic) shows that the plastic probe tip is not undergoing any noticeable deformation that may lead to loss in image resolution or z-direction accuracy. It should be noted that, due to the high smoothness of the mold (average roughness of approximately 2 nm), the surface of the polystyrene probe is smooth enough that a laser reflects adequately for signal acquisition without the necessity for metal coating (e.g., gold). By minimizing the imaging force the tip itself remained unaltered during imaging, thereby showing that the plastic tip can be used repeatedly without degrading its imaging quality.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A micro-cantilever, comprising:
a. a base;
b. a micro-cantilever beam, extending outwardly from the base, constructed from a material including a thermoplastic polymer material, the micro-cantilever beam having a distal end opposite the base; and
c. a tip extending transversely from the distal end, the tip constructed from a material including a thermoplastic polymer material and integrated with the micro-cantilever beam.

2. The micro-cantilever of claim 1, wherein the thermoplastic polymer material comprises a material selected from a group consisting essentially of: polystyrene, polypropylene, polyethylene, acrylonitrile butadiene styrene, polycarbonate, PMMA, polyester, polyimid, liquid crystal polymer, and polyamide.

3. A method of making a mold for a micro-cantilever, comprising the steps of:
a. selecting a geometry for a micro-cantilever beam having a distal end;
b. forming a mold defining a cavity having the geometry of the micro-cantilever beam; and
c. forming an indentation, corresponding to an image of a probe tip, in the mold at a selected location corresponding to the distal end of the micro-cantilever beam.

4. The method of claim 3, wherein the forming step comprises deforming a selected surface of the mold by pressing a tip into a selected portion of the mold at the selected location.

5. The method of claim 4, wherein the forming step further comprises using a nanoindenter to press the tip into the selected portion of the mold.

6. A method of making a micro-cantilever, comprising the steps of:
a. injecting a molten thermoplastic into a mold, the mold defining a cavity corresponding to a geometry of a cantilever beam having a distal end, the mold also defining an indentation disposed adjacent the distal end, the indentation corresponding to a geometry of a probe tip;
b. allowing the mold to cool so as to allow the molten thermoplastic in the cavity to substantially solidify, thereby forming a cantilever beam with a probe tip; and
c. removing the cantilever beam with the probe tip from the cavity.

7. The method of claim 6, wherein the thermoplastic comprises a material selected from a group consisting essentially of: polystyrene, polypropylene, polyethylene, acrylonitrile butadiene styrene, polycarbonate, PMMA, polyester, polyimid, liquid crystal polymer, and polyamide.
